(19) United States
(12) Patent Application Publication
(10) Pub. No.: US 2015/0219442 A1
(43) Pub. Date: Aug. 6, 2015

(54) LASER INSPECTION STATION

(71) Applicant: FORD MOTOR COMPANY, Dearborn, MI (US)

(72) Inventors: William Carl BUSHEY, Gibraltar, MI (US); Eric CZARNIOWSKI, Monroe, MI (US)

(73) Assignee: FORD MOTOR COMPANY, Dearborn, MI (US)

(21) Appl. No.: 14/174,078

(22) Filed: Feb. 6, 2014

(51) Int. Cl. G01B 11/14; G01V 8/10; G01N 33/26

(52) U.S. Cl. CPC G01B 11/14; G01N 33/26; G01V 8/10

(57) ABSTRACT

A laser inspection fixture for facilitating visual inspection of attachments to an object such as adhesive, sealant, wax or mastic daubs or lines on the article in a manufacturing process. The intended location of each object is illuminated by one laser that is provided with a dot profile template. The fixture may also be used to facilitate visual inspection of production tools and dies to verify tool set-up configurations.

### TECHNICAL FIELD

This disclosure relates to a multi-point inspection station for visually verifying the presence and location of paste-like materials, tool set-up pins, and other items that are applied or attached to an object.

### BACKGROUND

Visual inspection is one way of checking the location and presence of wax, sealer, adhesives fasteners, pins, or other objects that are applied, attached, or assembled in a production operation. A sample, exemplar, or template may be used as a visual guide for a visual inspection station. An inspector is expected to check the production part in a visual inspection in view of the sample part or template.

Sealers, adhesives, and wax are increasingly being used in vehicle assembly operations. These types of paste-like consistency materials are not as easy to check as a rivet or fastener that can be checked by sensors in the tool or by machine vision systems because they are precisely located and have a uniform appearance when installed. However, an adhesive deposit can flow or move as a result of gravity or forces exerted on the deposits as they are moved between stations or during application. The amount of lubricant or other coatings on a surface may also result in variation in adhesive location. These types of paste-like deposits do not always have the same appearance and are not easily checked with machine vision systems that are too sensitive which may result in excessive false alarms.

Vehicle doors, deck lids, hoods, and roofs may receive mastic, sealer, sound deadener, or wax applications in spots or lines that must be accurately applied to predetermined locations to be effective in joining outer body panels to inner panels and reinforcements. It is very difficult to visually verify the location and presence of a large number of deposits of such materials in a production environment without stopping the production line and using a tape measure to check the location of the deposits. If an adhesive or other application is missed, falls off, or is off location, subsequent remedial measures may need to be taken, at considerable expense, to correct the problem caused by the lack of the deposit in the proper location.

Visual inspection of die bolster pad pins and other tooling fixtures used in production operations is also difficult when a large number of points of inspection must be checked. After a die or fixture is set-up, a tedious verification of the tool set-up against tooling specification drawings must be performed to assure that the tool is properly set-up. For example, operation of a cushion die pad to produce parts may be impaired and may result in the improper production of parts if a die pad has a cushion pin that is placed in the wrong location or omitted.

This disclosure is directed to solving the above problems and other problems encountered in situations where visual inspections are employed as summarized below.

### SUMMARY

According to one aspect of this disclosure, a visual inspection fixture is provided for inspecting a plurality of locations on an object for the presence and location of a plurality of attachments. The fixture comprises a support frame and a plurality of lasers secured to the fixture that are each directed towards one of the plurality of locations to illuminate an area associated with each location.

According to other aspects of this disclosure, a controller selectively provides power to the lasers when a visual inspection is conducted. A plurality of dot profile templates are each attached to one of the lasers to project a line around each of the plurality of locations to facilitate a visual inspection for verifying that the attachments are all properly located on the object. The line may extend around each of the plurality of locations and may be enlarged to include an acceptable tolerance for the location of the attachments.

The support frame may include a plurality of parallel bars arranged side-by-side with the lasers being attached to the bars to be aimed at one of the plurality of locations. The lasers may be secured to the bars by an adjustable retainer that facilitates adjusting the location towards which each of the lasers are directed.

The attachment may be a line of material applied to the object, and the lasers may be directed to a plurality of locations along the line of material to facilitate a visual inspection of the location and presence of the line of material. Alternatively, the attachment may be a daub of paste-like material applied to the object. The material may be selected from a group including but not limited to adhesive, sealant, wax, and mastic.

In another application for the disclosed fixture, the object may be a tool that is adapted to receive a plurality of attachments selected ones of a plurality of locations. Each of the lasers may be directed towards one of the locations where one of the attachments are to be attached. In a specific application, the object may be a bolster plate for a die that includes a plurality of pockets, and wherein a sub-set of the pockets are selected to receive a press pin. Each one of the lasers is secured to the fixture to be directed towards one of the sub-set of the pockets to illuminate one of the pockets that was to receive one of the pins.

According to another aspect of this disclosure, a system is disclosed for visually inspecting a plurality of locations on an object. The system comprises projecting focused light in a pattern to illuminate each of the locations on the object. Each of the locations is visually inspected for the presence and the location of certain attachments that are intended to be disposed at each of the locations.

According to other aspects of the system the focused light may be the light from a laser. The light from the laser may be projected through a dot profile template to project a pattern of light that may form a line of light extending about the locations. The focused light may be provided by a plurality of lasers that each illuminates one of the locations on the object.

According to additional aspects of this disclosed system, the attachments may be daubs of paste-like material applied to the object. The material may be adhesive, sealant, wax, or mastic.

According to yet another aspect of this disclosure, a method is disclosed for visually inspecting an object that receives at least one attachment. The method comprises directing light focused on a plurality of locations on the object to provide illumination proximate the attachment at each of the plurality of locations. The attachments are visually inspected to determine the presence and the location of the attachments at each of the locations.

According to one application of the method, the attachment may be a daub of paste-like material applied to the object, and the light focused on the plurality of locations may be provided by a plurality of lasers that each illuminate one of the locations on the object.

According to another application of the method, the object may be a bolster plate for a die that includes a plurality of pockets. The attachment may be a press pin and a sub-set of the plurality of pockets may be selected to receive one of the press pins. The method may further comprise securing each of a plurality of lasers to a fixture to be directed towards one of the sub-set of pockets and illuminating each one of the pockets selected to receive one of the press pins. The step of visually inspecting may further comprise inspecting for the presence and the location of the press pins in the sub-set of pockets.

The above aspects of this disclosure and other aspects are described in greater detail below with reference to the attached drawings.

### BRIEF DESCRIPTION OF THE DRAWINGS

FIG. 1 is a perspective view of a laser visual inspection fixture;

FIG. 2 is a diagrammatic view of a laser and a dot profile template;

FIG. 3 is a fragmentary perspective view of an object, such as an inside surface of an outer panel for a vehicle having a plurality of daubs of a paste-like material applied in a predetermined pattern in locations highlighted by a set of rings of illumination;

FIG. 4 is a top plan view of a roof panel having a line of sealant applied around a skylight opening with dots of laser light projected adjacent the line of sealant;

FIG. 5 is a diagram of a press pin hole chart showing a desired arrangement of press pins in a die as they are specified to be assembled to a die bolster; and

FIG. 6 is a perspective view of a press including a die bolster having press pins installed in the bolster as the bolster is being inspected by the laser visual inspection fixture.

### DETAILED DESCRIPTION

A detailed description of the illustrated embodiments of the present invention is provided below. The disclosed embodies are examples of the invention that may be embodied in various and alternative forms. The figures are not necessarily to scale. Some features may be exaggerated or minimized to show details of particular components. The specific structural and functional details disclosed in this application are not to be interpreted as limiting, but merely as a representative basis for teaching one skilled in the art how to practice the invention.

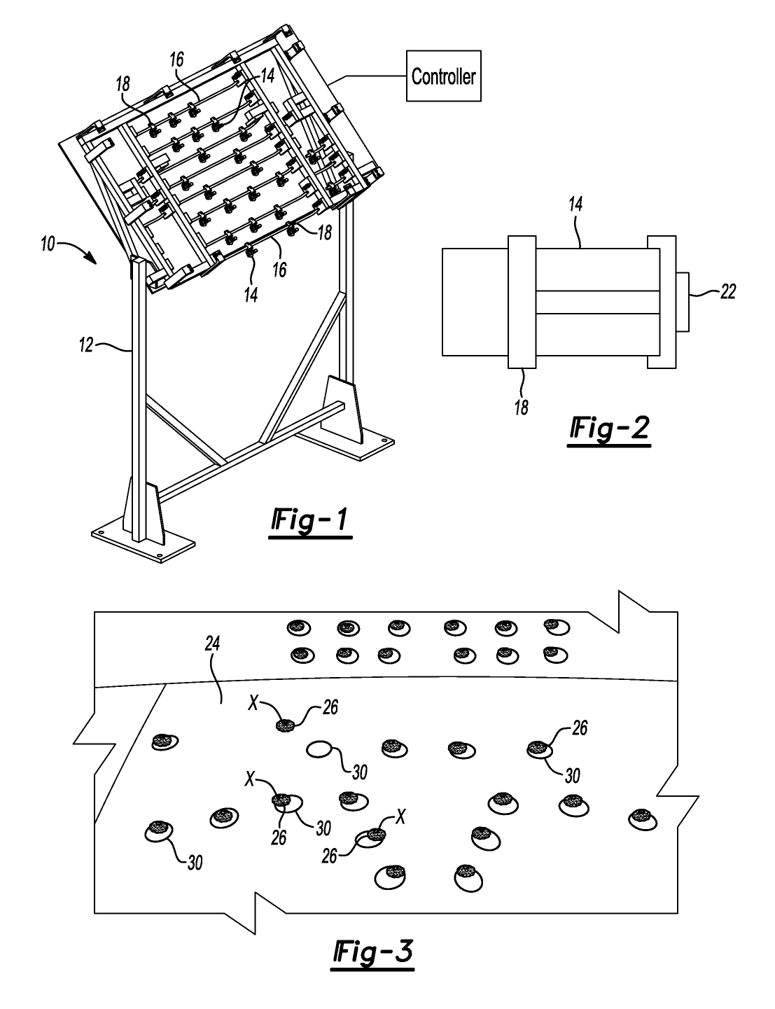

Referring to FIG. 1, a visual inspection fixture 10 includes a support frame 12. A plurality of lasers 14 are attached to a plurality of parallel bars 16 that are supported by the support frame 12. The lasers 14 are connected to the parallel bars 16 by adjustable retainers 18. The lasers 14 are directed to a predetermined location on an object and locked into place by the adjustable retainers 18. The visual inspection feature 10 is operatively connected to a controller 20. The controller 20 is used to turn the lasers 14 on to conduct a visual inspection of an object, as described below with reference to FIG. 3.

Referring to FIG. 2, a laser 14 is shown diagrammatically with a dot profile template 22 that limits the light emitted from the laser 14 to a circle of light or other shape corresponding to the shape of a desired attachment 26 (as shown in FIG. 3). The laser 14 is secured to the fixture (shown in FIG. 1) by an adjustable retainer 18.

Referring to FIG. 3, a section of a panel, such as a vehicle door, deck lid, roof or hood is illustrated with a plurality of attachments 26 disposed on the object 24. In FIG. 2, the illustrated attachments 26 are daubs of a paste-like material and may include adhesives, mastics, sealants, waxes and the like that are used in automotive component assembly operations. For example, adhesives may be used to augment other assembly techniques, such as welding, riveting and clinching. Adhesives are important in connecting inner panels and reinforcements to outer panels. The outer panels generally define class A surfaces that are painted and must be provided with a smooth surface that is not interrupted by attachment devices.

As shown in FIG. 2, the daubs of material or attachments 26 are shown within or at locations generally indicated by an illumination line 30. In FIG. 2, for example, several daubs 26 are shown partially outside of the illumination line 30. Some of the unacceptable daub locations 26 are indicated by an “X” in FIG. 2.

The illumination lines 30 are projected by the lasers 14 (shown in FIGS. 1 and 2) with one laser 14 being used to project each illumination line. In the illustrated embodiment, the illumination lines 30 are either oval or circular and are larger than the daubs 26. The illumination lines 30 are sized and shaped to include a tolerance area corresponding to the tolerance in the location of the daubs 26 that is added to the size of the intended daub of material.

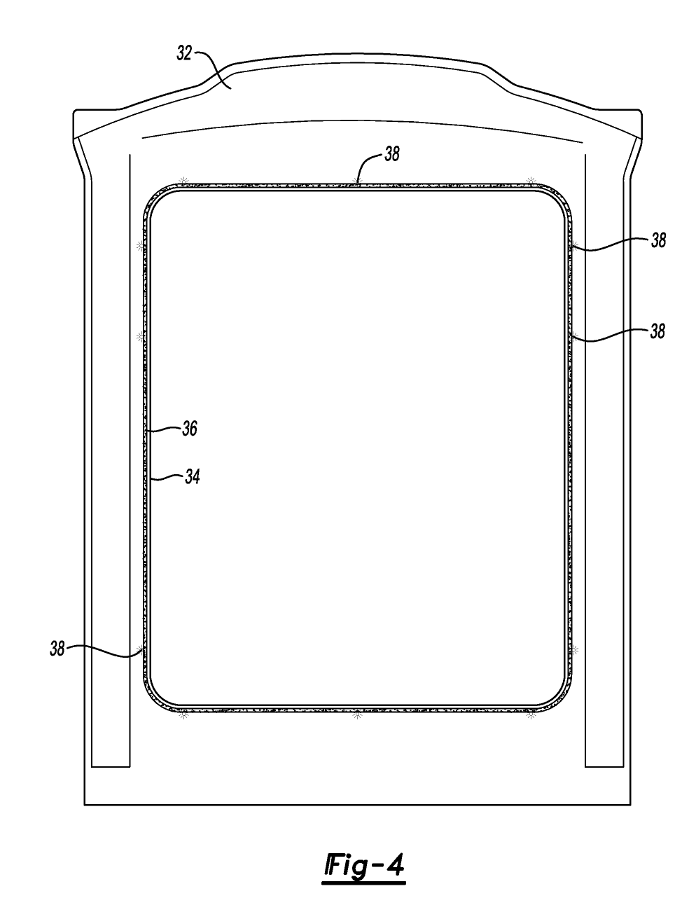

Referring to FIG. 4, a roof panel 32 is illustrated that defines a skylight opening 34. A sealant ring 36 is deposited around the periphery of the skylight opening 34 on the roof panel 32. In this particular inspection operation, an inspector visually inspects the location of the sealant ring 36 to be sure that it does not include any portion that extends too far outside of the skylight opening 34. A plurality of dot laser projections 38 are projected by the lasers 14 in selected locations where the alignment of the sealant ring 36 may be visually checked.

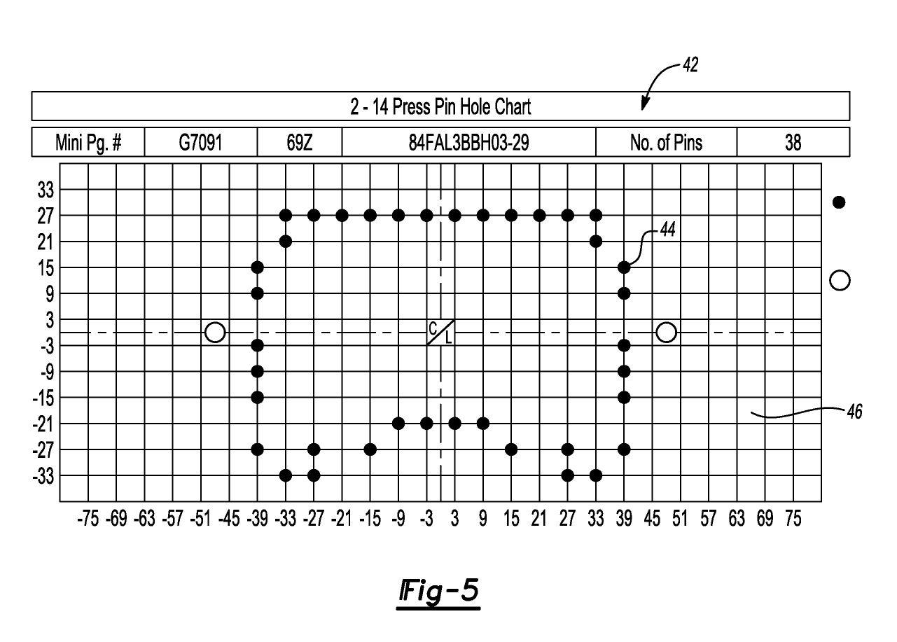

Referring to FIG. 5, a press pin hole chart 42 is provided that is used by die setters to set the location of press pins 44 on a cushion pad bolster 46 for a die before the die is loaded into a press for production purposes. The press pin hole chart includes, in the illustrated embodiment, 150 potential press pin locations along the X-axis and 66 press pin locations located along the Y-axis. The large number of potential press pin hole locations makes it time consuming and tedious to install the press pins 44 in the bolster 46 and then check to be sure that the press pins 44 are all located in the proper hole.

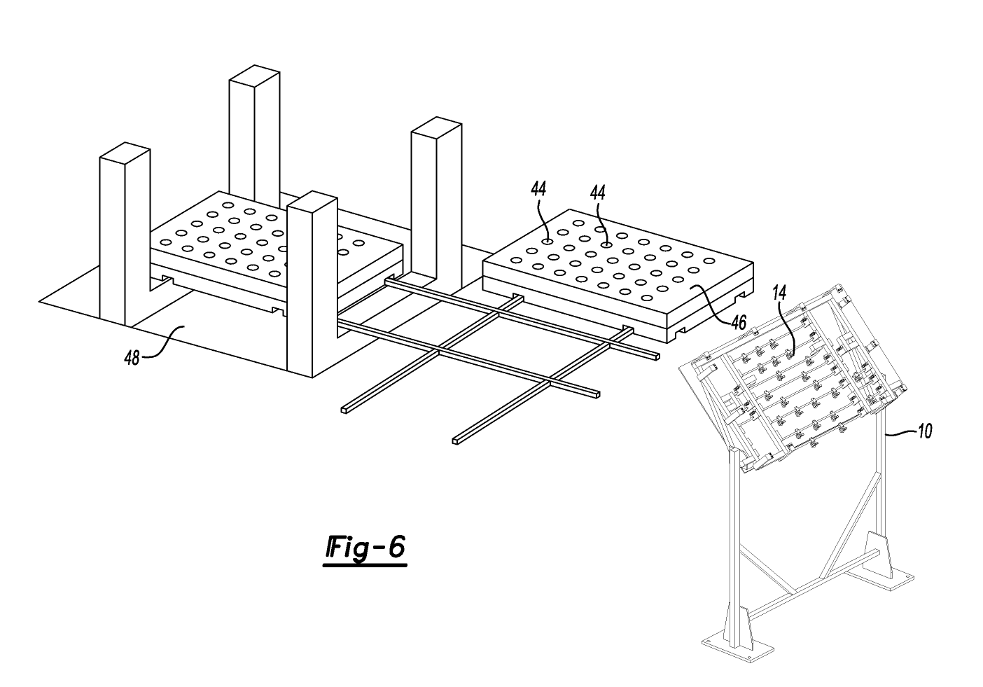

Referring to FIG. 6, a press pin 44 installation on a bolster 46 of a press cushion is shown in a position ready to be installed in a press 48. Prior to installing the bolster 46 with the press pin 44 in the press, a visual inspection fixture 10 may be used to check the location of the press pins 44. As described with reference to FIG. 1, the visual inspection fixture 10 includes a plurality of lasers 14 that are each projected on one of the intended press pin 44 installation hole locations. By projecting a beam of laser light onto the intended press pin locations, the press pin 44 installation can be visually checked with increased certainty.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A visual inspection fixture for inspecting a plurality of locations on an object for a presence and a location of a plurality of attachments, the fixture comprising:
a support frame; and
a plurality of lasers secured to the fixture that are each directed towards one of the plurality of locations to illuminate at least part of each location.

2. The fixture of claim 1 further comprising:
a controller for selectively providing power to the lasers when a visual inspection is conducted.

3. The fixture of claim 1 further comprising:
a plurality of dot profile templates that are each attached to one of the lasers to project a line around each of the plurality of locations to facilitate a visual inspection for verifying that the attachments are all properly located on the object.

4. The fixture of claim 3 wherein the line extends around each of the plurality of locations including an acceptable tolerance for the location of the attachments.

5. The fixture of claim 1 wherein the support frame includes a plurality of parallel bars arranged side-by-side, and wherein the lasers are attached to the bars to be aimed at one of the plurality of locations.

6. The fixture of claim 5 wherein the lasers are secured to the bars by an adjustable retainer that facilitates selective modification of the location that each of the plurality of lasers are each directed towards.

7. The fixture of claim 1 wherein the attachment is a line of material applied to the object, and wherein the lasers are directed to a plurality of locations along the line of material to facilitate a visual inspection of the presence and the location of the line of material.

8. The fixture of claim 1 wherein the attachment is a daub of paste-like material applied to the object.

9. The fixture of claim 8 wherein the material is selected from a group consisting essentially of:
adhesive;
sealant;
wax; and
mastic.

10. The fixture of claim 1 wherein the object is a tool that is adapted to receive a plurality of attachments each in one of a plurality of locations, and wherein each of the lasers is directed towards one of the locations where one of the attachments are to be attached.

11. The fixture of claim 1 wherein the object is a bolster plate for a die that includes a plurality of pockets, and wherein a sub-set of the pockets are selected to receive a press pin, and wherein each one of the lasers is secured to the fixture to be directed towards one of the sub-set of the pockets to illuminate one of the pockets selected to receive one of the pins.

12. A system for visually inspecting a plurality of locations on an object comprising:
projecting focused light in a pattern to illuminate each of the locations on the object; and
visually inspecting each of the locations for a presence and a location of attachments at each of the locations.

13. The system of claim 12 wherein the focused light is light from a laser.

14. The system of claim 13 wherein the light from the laser is projected through a dot profile template, and wherein the pattern is a line of light extending about the locations.

15. The system of claim 12 wherein the focused light is provided by a plurality of lasers, and wherein each laser illuminates one of the locations on the object.

16. The system of claim 12 wherein the attachment is a daub of paste-like material applied to the object.

17. The system of claim 16 wherein the material is selected from a group consisting essentially of:
adhesive;
sealant;
wax; and
mastic.

18. A method of visually inspecting an object that receives at least one attachment comprising:
directing light focused on a plurality of locations on the object to provide illumination proximate the at least one attachment at each of the plurality of locations; and
visually inspecting a presence and a location of the at least one attachment at each of the plurality of locations.

19. The method of claim 18 wherein the at least one attachment is a daub of paste-like material applied to the object, and wherein the light focused on the plurality of locations is provided by a plurality of lasers that each illuminate one of the locations on the object.

20. The method of claim 18 wherein the object is a bolster plate for a die that includes a plurality of pockets, wherein the attachment is a press pin, and wherein a sub-set of the plurality of pockets are selected to receive one of the press pins, and wherein the method further comprises:
securing each of a plurality of lasers to a fixture to be directed towards one of the sub-set of the plurality of pockets;
illuminating each one of the pockets selected to receive one of the press pins; and
wherein the step of visually inspecting further comprises inspecting the presence and location of the press pins in the sub-set of the plurality of pockets.
